(19) United States
(12) Patent Application Publication
(10) Pub. No.: US 2023/0049226 A1
(43) Pub. Date: Feb. 16, 2023

(54) INJECTOR GUARD AND METHOD OF USE

(71) Applicant: Anthony E Angelillo, Wethersfield, CT (US)

(72) Inventor: Anthony E Angelillo, Wethersfield, CT (US)

(21) Appl. No.: 17/399,514

(22) Filed: Aug. 11, 2021

(51) Int. Cl. A61M 37/00

(52) U.S. Cl. CPC A61M 37/0076; A61M 2205/273; A61M 2210/04

(57) ABSTRACT

An injector guard and related method of use may be used for micropigmentation and other similar procedures. The guard may attach to a removable or disposable component, such as an ink cartridge, or may connect to a reusable device, such as a tattoo gun. The guard may be also unitary with the ink cartridge, needle, or micropigmentation device. It may be disposable or may be reusable. It may include a leg which may be used to aid in controlling the position of the micropigmentation tool as well as the depth of penetration of the needle into the dermis, as well as, optionally, the angle of the instrument. The guard may also include a coupling body, which may attach to an ink cartridge or other device or component by a press-fit, snap-fit, friction fit, threading, or other means.

### BACKGROUND

The present disclosure relates generally to an injector guard device and method of use. More particularly, the present disclosure relates to an injector guard device which may be used as part of a system with micropigmentation equipment or may itself be a feature of such equipment, and associated methods of use.

Micropigmentation is a form of what is commonly referred to as “permanent makeup”. It is also commonly referred to as dermapigmentation or cosmetic tattooing. Micropigmentation uses permanent or semi-permanent pigmentation of the dermis, also known as “tattooing”, to leave desired marks and colors on the skin. Micropigmentation may be used to disguise what an individual may consider to be imperfections in their appearance, or it may be used to enhance their appearance to desired levels. In one application of micropigmentation, an individual may desire to have the appearance of additional hair follicles, or a different pattern, density, or coloring of hair follicles. Common applications of micropigmentation to simulate hair follicles include eyebrows, such as for those who have lost their eyebrows due to medical reasons or simply desire fuller or differently shaped eyebrows, as well as hairlines, hair density, and undesirably bald areas. One who applies micropigmentation, such as a tattoo artist or a micropigmentation technician or specialist, may create the appearance of hair follicles on an individual through the use of micropigmentation, applying substances such as tattoo ink to the dermis or below the dermis at desired depths, desired angles, in desired patterns, and in desired colors.

Various devices and methods related to micropigmentation are known in the art. Most means of applying micropigmentation involve the use of one of a number of types of tools, such as tattoo guns, which use a needle of varying gauges to apply the pigmentation. Needles, as well as the associated cartridges and also ink/pigmentation reservoirs, are typically disposable, with a new one being used for each customer, thus reducing the risk of cross-contamination between customers, or whenever previous components otherwise require replacement. The applicator is commonly handheld, requiring the micropigmentation specialist to maintain position, angle, and depth of needle penetration completely by hand and by eye.

A common problem with micropigmentation is for the user to maintain even, consistent, and desired distance of the needle and the micropigmentation tool relative to the dermis. Inconsistent height, as well as angle and shaking of the tool, can lead to blowouts and micropigmentation results which are not uniform, including cosmetic hair follicles which do not look realistic. Additionally, excessive depth and prolonged time in a given region while attempting to create consistent marks or lines can lead to migration of ink, scars, skin cracking, blistering, infection, and other cosmetic and medical concerns. Swollen tissue from too much time spent in a single area, which is further overworked, can lead to even greater migration of ink.

Current micropigmentation devices and methods of use continue to lead to imperfect results even in the hands of skilled micropigmentation specialists. Current micropigmentation devices do not adequately enable steady and even application of micropigmentation at controlled and consistent depths relative to the dermis.

### SUMMARY

The present disclosure relates generally to an injector guard device and method of use. More particularly, the present disclosure relates to an injector guard device which may be used as part of a system with micropigmentation equipment or may itself be a feature of such equipment, and associated methods of use. The injector guard device enables a user to effectively, consistently, and repeatably apply micropigmentation at a desired depth while minimizing time at any given area of the dermis, while also at least partially stabilizing the device relative to the dermis.

The injector guard may include a leg which extends downward from a top portion towards a bottom portion. There may optionally also be a connecting body which, in some embodiments, may have a top, bottom, an outer surface, and an inner surface. The connection body may be configured to connect to a component of a pigmentation device, such as a tattoo gun or its commonly associated components or attachments. This device may include a needle, such as one which is a part of a cartridge or is otherwise a disposable component. The component to which the injector guard attaches or of which it is itself a further feature or component may be disposable or it may be reusable. The injector guard, in some embodiments, may attach directly to a needle cartridge, to an ink cartridge, or it may attach to a tattoo gun or other micropigmentation device or component of a device.

Throughout this disclosure, needles may be referred to in the singular or in the plural. It should be understood that, even when referred to as a single needle, a plurality of needles may be used, such as common tattooing cartridges that include multiple needles in different groupings or relative orientations, as common tattoo “needles” do in fact often comprise multiple needles which may be attached or removed as a group.

The leg of the injector guard may have a bottom surface, or foot, which is closer to the top of the injector guard than the penetrating tip of the needle is. In this way, the foot of the injector guard may act as a depth stop, coming in contact with the dermis and having the tip of the needle extend a predetermine distance below the foot, and thus to a predetermined penetration into the dermis.

In embodiments where a connecting body is present, the leg may have a feature where at least part of the leg extends outwards and away from the connecting body. In some embodiments, the leg may additionally or alternatively have a feature where at least some of the leg extends inwards from the connecting body, such as towards the needle.

In some embodiments, the connecting body, where present, may be substantially circular when viewed from above. In other embodiments, the connecting body may be elliptical but not circular, deforming into a circular cross-section when pressed onto the cartridge or other component to which the injector guard is attached. Other shapes may also be used, such as octagons, hexagons, rectangles, triangles, hybrid shapes, irregular shapes, and other shapes.

In embodiments with a connecting body, the connecting body may optionally have an opening. This opening may allow for the connecting body to flex open to receive its connecting component, such as a cartridge, or may allow flexion for other mating features to engage, such as a rib which may have to pass into a complementary slot or which might need to pass over a rib or other feature on the mating component. As mentioned above, the connecting body, in embodiments where it is present, may optionally include a rib, a recess, or other connection feature. This connection feature, when interacting with a complementary feature on a tattooing cartridge or other component, may hinder the injector guard from sliding down and off of the cartridge or other component, may resist rotation relative to that component, or both. In other embodiments, the connecting body may include a threaded or partially threaded region or feature to aid in retention of the injector guard to the cartridge or other component, or that other component may include a threaded or partially threaded feature which may interact with a feature on the injector guard to aid in its relative retention.

In some embodiments, the injector guard may include a wider area at the foot of the leg. This wider area may be a foot which is entirely larger than the typical width of the injector guard above it, may be a rib feature, or may be one or more protrusions extending from the foot at one or more sides. The leg may also include one or more ribs extending along the height, width, or at an angle along the leg so as to improve rigidity of the leg and resist flexion during use.

The injector guard may be part of a kit, a set, or an assembly, which may include the injector guard, a cartridge, a tattoo gun or similar micropigmentation device, and one or more needles, which may be part of one of the other components or may be a separate component, such as where the one or more needles are disposable and replaceable. The injector guard may be a separate component, such as where it is removable, or may be unitary with the cartridge or with the tattoo gun or similar micropigmentation device.

In one method of use, a user may attach a cartridge to a tattoo gun or similar micropigmentation device. The user may press an injector guard against the dermis of an individual, and then tattoo or otherwise apply pigment to that individual, penetrating the dermis of the individual with a needle. As an additional step, where the injector guard is a separate component, the user may attach the injector guard to the cartridge, or the user may attach the injector guard to the tattoo gun or other similar micropigmentation device.

The disclosure relates to an injector guard and an improved micropigmentation application device and method of use, such as through the use of the described invention. The method may include the use of an injector guard which may include one or more of the features or components described herein. Any number of components or features may be used in conjunction with and incorporated into the injector guard and the micropigmentation device described. Various materials, including metals, plastics, and composites, may be used for the described components and features.

It is to be understood that the above mentioned features and the features yet to be explained hereinafter can be used not only in the respectively mentioned combinations but also in other combinations or alone without departing from the context of the present invention.

### BRIEF DESCRIPTION OF THE DRAWINGS

The invention is now disclosed in detail with reference to exemplary embodiments shown in the accompanying drawings, where:

FIG. 1 shows a front top right isometric view of an embodiment of an injector guard device of the present disclosure;

FIG. 2 shows a front bottom left isometric view of the embodiment of the injector guard device of FIG. 1;

FIG. 3 shows a front view of the embodiment of the injector guard device of FIG. 1;

FIG. 4 shows a top view of the embodiment of the injector guard device of FIG. 1;

FIG. 5 shows a bottom view of the embodiment of the injector guard device of FIG. 1;

FIG. 6 shows a front top right isometric view of the embodiment of the injector guard device of FIG. 5 connected to a cartridge;

FIG. 7 shows a front view of the embodiment of the injector guard device connected to a cartridge of FIG. 6;

FIG. 8 shows a front view of an alternate embodiment of the injector guard device of the present disclosure which is unitary with a cartridge;

FIG. 9 shows a front view of another embodiment of the injector guard device of the present disclosure;

FIG. 10 shows a front top right isometric view of an alternate embodiment of the injector guard device of the present disclosure;

FIG. 11 shows a top view of the embodiment of the injector guard device of FIG. 10;

FIG. 12 shows a front top right isometric view of another embodiment of the injector guard device of the present disclosure;

FIG. 13 shows a top view of the embodiment of the injector guard device of FIG. 12;

FIG. 14 shows a front view of the embodiment of the injector guard device of FIG. 12;

FIG. 15 shows a front top right isometric view of an alternate embodiment of the injector guard device of the present disclosure;

FIG. 16 shows a front cross-sectional view of the embodiment of the injector guard device of FIG. 15;

FIG. 17 shows a front top right isometric view of another embodiment of the injector guard device of the present disclosure;

FIG. 18 shows a front cross-sectional view of the embodiment of the injector guard device of FIG. 17;

FIG. 19 shows a front top right isometric view of another embodiment of the injector guard device of the present disclosure; and

FIG. 20 shows a front top left isometric view of an alternate embodiment of the injector guard device of the present disclosure.

### DETAILED DESCRIPTION

Exemplary embodiments of the disclosure are illustrated in the Figures and are explained in the following description in more detail, wherein identical reference numbers refer to identical, or similar, or functionally identical or similar components.

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features or those previously described are omitted or simplified in order not to obscure the illustrative embodiments.

Various operations may be described as multiple discrete operations, in turn, in a manner that is most helpful in understanding the present invention, however the order of description should not be construed as to imply that these operations are necessarily order dependent. In particular, these operations need not be performed in the order of presentation. In some instances, structures and devices may be shown in block diagram or flow chart form in order to facilitate describing the disclosed subject matter.

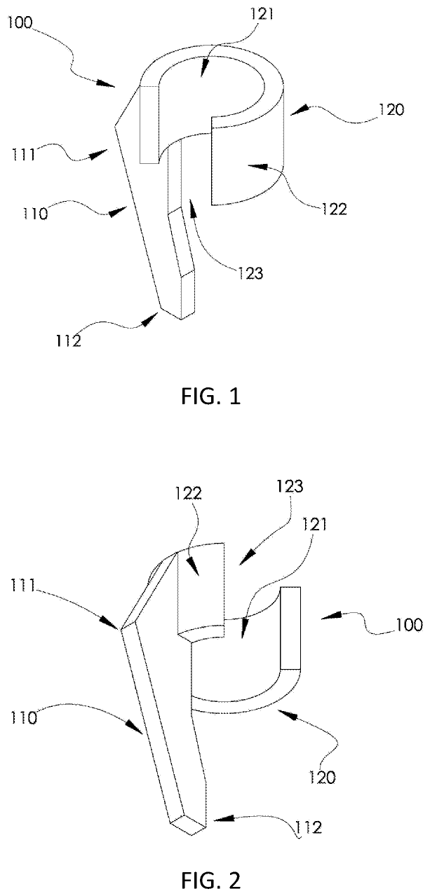

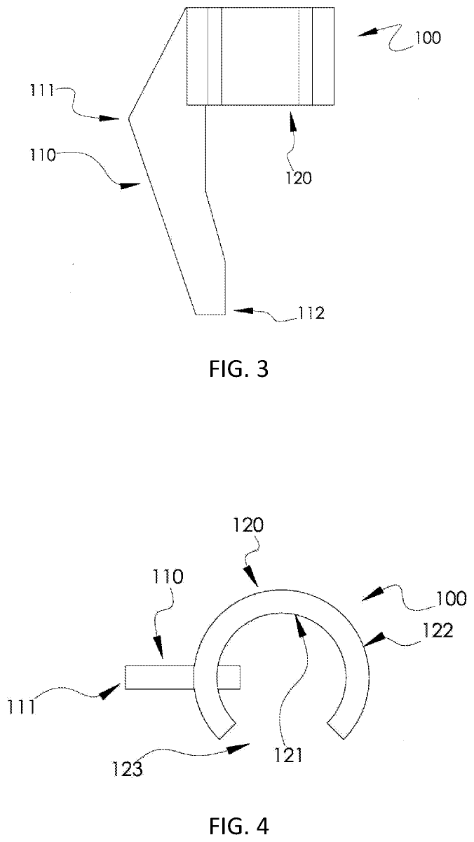

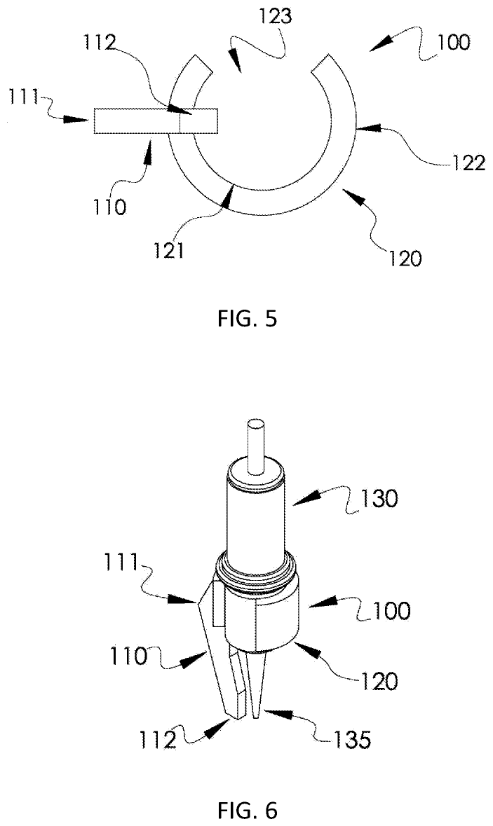

An embodiment of the injector guard 100 of the present disclosure is shown in FIGS. 1-5. The injector guard 100 may be used in combination with a tattooing or micropigmentation application device to aid in maintaining the distance and penetration of a needle used in the application of pigmentation, such as to the dermis, relative to the dermis. The injector guard 100 may be made of any material with suitable rigidity, may be at least partially flexible, and may include additional elements or materials, such as those for cushioning contact points. The injector guard 100 may be made of materials such as plastic, metal, resin, or hybrid materials. The injector guard 100 may be unitary, or it may be made up of a plurality of sub-components. The injector guard 100 may be molded, such as by injection molding, may be machined, such as from larger pieces of stock material or from partially finished blanks, may be made through additive manufacturing, such as 3-D printing, or may be made by other means known in the art. The injector guard 100 may be configured to be disposable, may be configured, made of a material, or surface-coated so as to be able to be sterilized, cleaned, or otherwise enabled for repeated use. The injector guard 100 may also optionally be configured to be covered by, connected to, or otherwise to come in contact with a protective material, such as a clear plastic layer of separate material, which may create a sterile or other hygienic barrier between the injector guard 100 and an individual to whom micropigmentation is being applied or between the injector guard 100 and the operator of the micropigmentation device. The injector guard 100 may also optionally include an absorbent material or other dabbing feature, configured to avoid smearing of ink where the injector guard 100 may come into contact with the dermis.

The injector guard 100 may include a leg 110 which may optionally extend from a connection body 120 to a distal end of the injector guard 100, wherein the distal end would be proximate to the dermis of an individual who is receiving micropigmentation. The connection body 120 may be of various shapes, with an inner profile 121 generally configured to at least partially conform to those of one or more ink cartridges. In the embodiment shown, the inner profile 121 is shown as being substantially circular, though it should be understood that other shapes of the inner profile 121 may also be used without deviating from the scope or intent of this disclosure. The inner profile 121 of the connection body 120 may be substantially circular, elliptical, rectangular, triangular, pentagonal, hexagonal, octagonal, a hybrid shape, or other shapes. The inner profile 121 of the connection body 120 may be configured to retain itself, and the injector guard 100, in connection with a cartridge by means of a friction fit, such as where the inner profile 121 is circular or elliptical, or may be at least partially oversized relative to the cartridge but include nubs or other contact or retention features to create a friction fit, press fit, snap fit, or other means of retention known in the art. The connection body 120 may be configured to removably connect to the cartridge, may be configured to permanently connect to the cartridge, or may be unitary with the cartridge. The outside profile 122 of the connection body 120 may be substantially similar to that of the inside profile 121, or may be a different shape or have varying thicknesses relative to the inside profile 121, or may include additional features, recesses, or protuberances.

In the embodiment shown, the connection body 120 includes an attachment opening 123 configured to allow passing the connection body 120 around a portion of the cartridge. The connection body 120 may be made of an at least partially flexible material, where the connection body 120 flexes or otherwise elastically or partially plastically deforms to permit connection of the connection body 120 to a portion of a cartridge. The connection body 120 then returns to its original shape, or at least close enough to that original shape to ensure connection is retained. The shape and sizing of the attachment opening 123, as well as the material selection and wall thicknesses of the connection body 120, may be configured to enable removable connection of the injector guard 100 to a cartridge, or may be configured to enable permanent connection of the injector guard 100 to a cartridge. It should be understood that, though this disclosure primary speaks of connection of the injector guard 100 to a cartridge, other embodiments may include connection of the injector guard 100 to the micropigmentation device instead, whether removably or permanently, or the injector guard 100 may be unitary with either the cartridge of the micropigmentation device, without deviating from the scope or intent of this disclosure.

The leg 110 may be substantially rectangular in cross-section, circular, triangular, of other shapes known in the art, may be a hybrid shape, may have recesses or protuberances, or may vary in cross-section along the length of the leg 110. From a side perspective, the leg 110 may be substantially uniform, such as being rectangular shaped, may be triangular, may be a hybrid shape, may include multiple straight edges, and may include one or more curves. In the embodiment shown, the leg 110 includes at least one prominence 111 which may extend along at least a portion of the leg 110 away from the connection body 120. The prominence 111, as shown, is angled and triangular, though other shapes as well as curves or radiused features may be used without deviating from the scope of intent of this disclosure. The prominence 111 may be configured to provide a feature to aid in gripping of the injector guard 100 or associated cartridge or micropigmentation device by a user, may aid in alignment of one or more of those components or devices, or may aid in enabling a user to identify the location of the leg 110 of the injector guard 100 when it might otherwise be obscured by a user's fingers or hand. The prominence 111 may also be configured so as to strengthen the leg 110, whether in relation to compression, flexion, or both.

The leg 110 may include a foot feature 112. The foot feature 112 may extend in and past the inner profile 121, such as towards a needle of a cartridge, may be substantially in line with at least a portion of the inner profile 121, or may be outside of the inner profile 121, relative to a top-down view of the injector guard 100. The foot feature 112 may be substantially flat, may be curved, and may include other features or materials, such as for dabbing ink. The foot feature 112 may be of a substantially uniform thickness relative to other portions of the leg 110, may be wider than other portions of the leg 110, such as to provide greater support of the micropigmentation device through the injector guard 100, or may be thinner that other portions of the leg 110, such as to minimize contact of the foot feature 112 with the dermis.

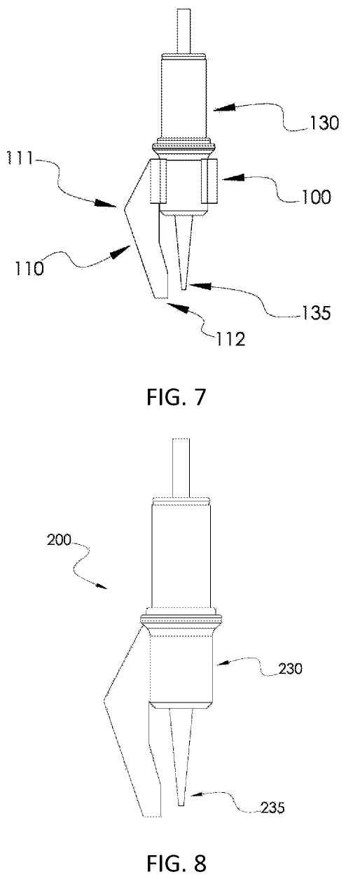

An injector guard 100 connected to a cartridge 130 with a needle 135 is shown in FIGS. 6-7. The injector guard 100 may slide over a portion of the cartridge 130 to enable connection or may require deflection of at least a portion of the connection body 120 to enable attachment, such as where a portion of the cartridge 130 passes through the attachment opening 123 of the injector guard 100. Some types of cartridges 130 may allow for sliding the cartridge through the inner profile 121 of the injector guard 100, while others, such as those which may have prominences or ribs along the length of the cartridge 130 may require other means of attachment, such as at least partial deflection of the connection body 120 of the injector guard 100 and creation of a snap-fit-like connection.

Another embodiment of the injector guard 200 is shown in FIG. 8. In the embodiment shown, the injector guard 200 is unitary with the cartridge 230 and the needle 235. In such an embodiment, the complete injector guard 200, with cartridge 230 and needle 235, may be provided in a sterile condition, such as through sterilization and sterile packaging, and then may be disposed of all together.

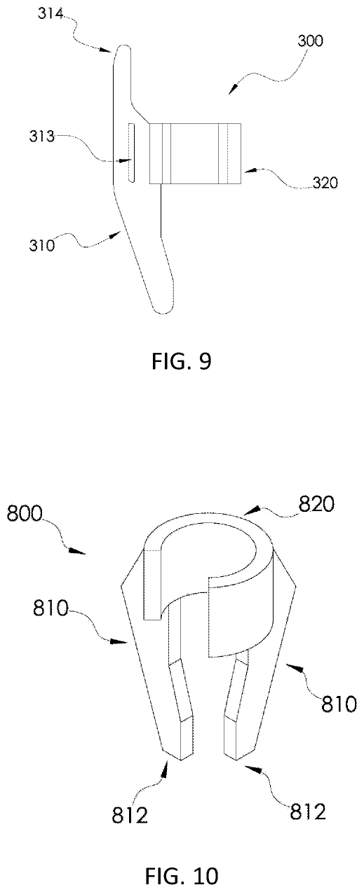

The injector guard 300 may include one or more openings, windows, or slots in the connection body 320, the leg 310, or both. In the embodiment shown in FIG. 9, the injector guard 300 includes an opening 313 in the leg 310. One or more openings 313 or windows may be included to reduce weight of the injector guard 300, improve in visibility when using a micropigmentation device with a connected injector guard 300, or for other reasons which may be beneficial to a user. Other shapes and sizes of openings 313 may also be used, or quantities of openings 313, such as to create substantial visibility through the leg 310 of the injector guard 300.

In the embodiment shown, the injector guard 300 includes an extension 314. The extension 314 may optionally extend from the connection body 320, from the leg 310, or from other features of the injector guard 300. The extension 314 may be configured to aid in retention or alignment of the injector guard 300 and of the micropigmentation device in the hand of a user, may be configured to more securely attach the injector guard 300 to either the cartridge, the micropigmentation device, or both, or may be configured to provide a feature to aid a user in removing the injector guard 300 from the cartridge or micropigmentation device, or in removing both the injector guard 300 and the cartridge together from the micropigmentation device for cleaning or disposal.

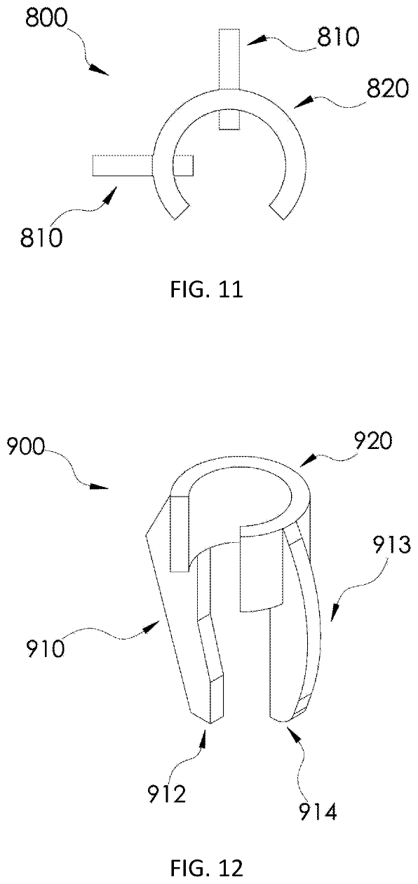

Another embodiment of the injector guard 800 is shown in FIGS. 10-11. The injector guard 800 may include a plurality of legs 810 which may optionally extend from the connection body 820. In the embodiment shown, the injector guard 800 includes two legs 810, which are set along the connection body 820 at an angle of 90° relative to each other, from a top-down perspective. It should be understood that other quantities of legs can be used, other angular offsets may be used, and, where more legs 810 are present, the relative angles between any pair of legs 810 may vary relative to any other pair of legs 810. Each leg 810 may include a foot 812, wherein multiple feet 812 may be substantially in the same plane with each other, or the feet 812 may be of different shapes or may be on legs 810 of different heights. In some embodiments, the relative angle between legs 810 may allow for improved visibility of the needle by a user, thumb placement by a user, and angling of the instrument. In the embodiment shown, relative to a top-down view, one leg 810 is at a 9 o'clock position while the other leg 810 is at a 12 o'clock position, enabling significant visibility by a user of the needle along the clockwise range between the 12 o'clock position and the 9 o'clock position, as well as allowing for improved angling of the device while tilting towards that range.

FIGS. 12-14 show an alternate embodiment of the injector guard 900. The injector guard 900 may optionally include a plurality of legs 910, 913, which may be set at different locations along the connection body 920. The legs 910, 913 may be of different heights, thus one leg 910 may contact the dermis before the other leg 913 does. With one leg 913 being shorter than another leg 910, a user may make contact with the dermis while using the other leg 913 to aid in gripping the injector guard 900, as a further backstop, or for controlled angling of the injector guard 900.

In the embodiment shown, one leg 910 has a foot 912 which is substantially flat, while the other leg 913 has a foot 914 that includes at least one curved surface, aiding in using that foot 914 for rolling the injector guard 900 along the dermis. It should be understood that either foot 912, 914 may be substantially flat, may be curved or have curved features, or a combination may exist without deviating from the scope or intent of this disclosure.

One foot 912 may be closer to the needle than another foot 914, or one leg 910 may have at least one surface or feature which may be closer to the needle than features or surfaces of the other leg 913. In the embodiment shown, one leg 910 and its associated foot 912 include features and surfaces which extend closer to the needle than any surfaces or features of the other leg 913 or its associated foot 914. It may be beneficial to have at least one foot 912 close to the needle as, the closer that a foot 912 is to the needle the less penetration variability there is with what slight angling of the injector guard 900 may be present during use or during attachment to other components or instruments. The closer that the foot 912 is to the needle, the more consistent and repeatable the depth of penetration into the dermis is likely to be during typical use. In the embodiment shown, the foot 914 of the other leg 913 is spaced further away from the needle to allow for improved visibility of the needle during use.

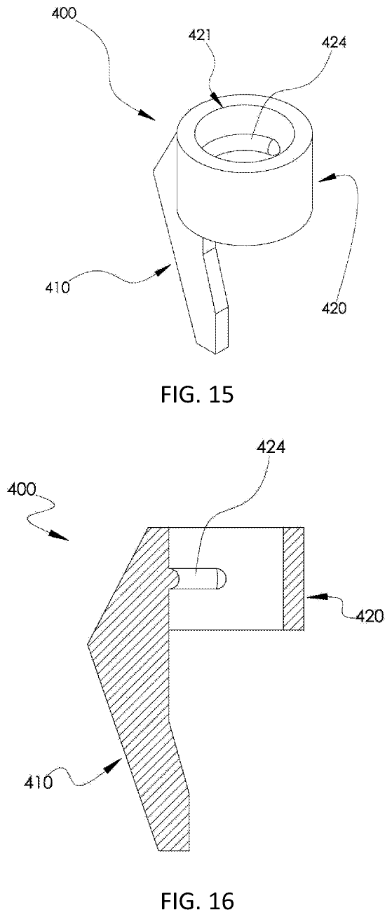

Another embodiment of the injector guard 400 is shown in FIGS. 15-16. The injector guard 400 may optionally include a rib 424 or other connection feature, such as a prominence or recess, to aid in connecting to an ink cartridge or micropigmentation device. The rib 424 or other connection feature may be a feature of, connected to, or extending from the connection body 420, as shown in this embodiment, or may be located on or at other features, such as the leg 410. The rib 424 may extend substantially around the circumference of the inner profile 421 of the connection body 420 or substantially over the length of whichever surface it extends from, or may only go partially around or on that surface, such as with the rib 424 shown. The rib 424 may be configured to at least partially fit into a complementary recess in a cartridge or in the micropigmentation device, such as for attachment to components which may have at least one complementary recess or where those components may have their own rib or feature which, in combination with the injector guard 400 rib 424, would resist movement of the injector guard 400 relative to a cartridge or micropigmentation device. It should be understood that, in embodiments where the injector guard 400 is configured to attach to a cartridge or micropigmentation device which may have a protrusion, the injector guard 400 may have a complementary recess to receive at least a portion of that protrusion and thus resist relative motion or disassociation.

In this embodiment, the rib 424 has a substantially curved surface, to aid in attachment to a cartridge or micropigmentation device, as well as optional removal, though other shapes and contours may also be used without deviating from the scope or intent of this disclosure. In some alternate embodiments, the rib 424 may have at least one sharp corner, thus creating a snap-fit attachment to the cartridge for easy removal and disposal of the injector guard 400 and a cartridge as a single unit. Though not shown in this embodiment, it should also be understood that the connection body 420 may also have either an opening or a decrease in wall thickness in at least one area to aid in flexion of the connection body 420, thus enabling connection of the injector guard 400 to a cartridge or micropigmentation device over the rib 424 or any prominent features of the cartridge or micropigmentation device.

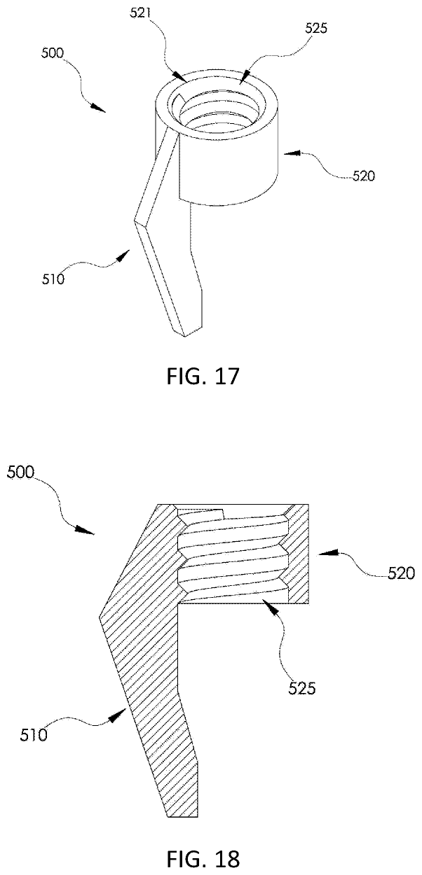

FIGS. 17-18 show another alternate embodiment of the injector guard 500. The injector guard 500 may connect, removably or permanently, to the cartridge or to the micropigmentation device. In the embodiment shown, the injector guard 500 includes a threaded feature 525. The cartridge or the micropigmentation device may include a complementary threaded feature, or may include a protrusion or recess with which the threaded feature 525 of the injector guard 500 is configured to interact, such as a partial rib. The threaded feature 525 of the injector guard 500 may be located along the inner profile 521 of the connection body 520 of the injector guard in some embodiments. In other embodiments, the cartridge or the micropigmentation device may include a threaded feature, and the injector guard 500 may include only a partially threaded feature 525, or may only include a recess or protrusion configured to interact with the threaded feature of the cartridge or the micropigmentation device to resist axial motion of the injector guard 500 relative to an axis passing through a needle of the cartridge. In such embodiments, the partially threaded feature 525 or protrusion or recess of the injector guard 500 may be located on the connection body 520, on the leg 510, or on an extension of the injector guard 500, if present.

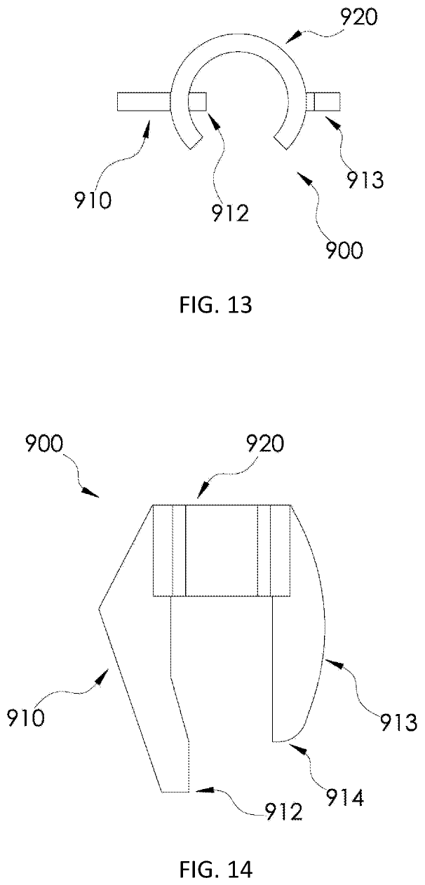

It should be understood that, though the threaded feature 525 of the injector guard 500 is shown as a full thread in FIGS. 12-13, if may also only be a partial thread, and may also not extend along the full height of the feature of the injector guard 500 on which it is located without deviating from the scope or intent of this disclosure.

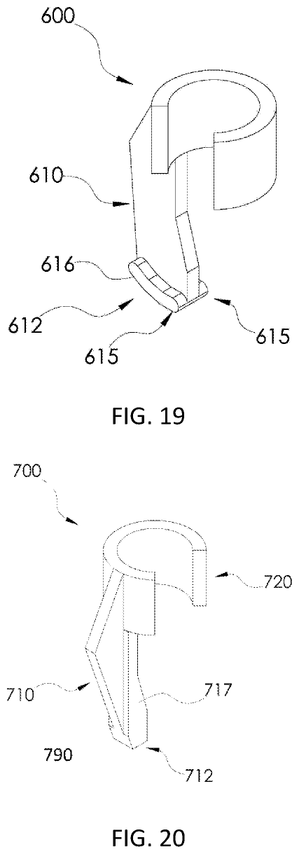

Another embodiment of the injector guard 600 is shown in FIG. 19. In this embodiment, the foot 612 of the injector guard 600 is wider than at least some other portions of the leg 610. The foot 612 includes one or more ridges 615 which widen the foot 612. The ridges 615 create a larger footprint to the leg 610 where it potentially contacts the dermis, allowing for a thin leg 610 while giving a substantial foot 612 which may give a user a more positive sense of contact with the dermis, may aid in keeping a user's fingers out of the way, and may ensure that a user has the needle entering the dermis at the correct and desired angle, as opposed to holding the instrument at a slight angle, while unwittingly having only an edge of the foot 612 contacting the dermis, and thus not having the needle penetrate the dermis to the intended depth. It should be understood that, in other embodiments, one or more of the ridges 615 may extend up at least a portion of the leg 610, thus giving improved strength against flexion of the leg 610.

In the embodiment shown, the foot 612 of the injector guard 600 also includes one or more angled surfaces 616 proximate to where the leg 610 contacts the dermis. These angled surfaces 616 may be located on the foot 612, on the leg 610 itself, or on both. The angled surfaces 616 may be set at predetermined angles relative to the surface of the foot 612 which contacts the dermis, such as 15°, 30°, 45°, 60°, 75°, or at any angle which may be beneficial to a user. Different injector guards 600 may also be made available, or included in a kit, with different angled surfaces 616 for different user preferences, different methods of micropigmentation, or other varying needs. A user may choose to tilt the injector guard 600 at varying angles for different types of pigmentation application, different desired dispersals of pigmentation, or to achieve different depths of penetration into the dermis, where it may be known what results different angles of angled surfaces 616 will achieve. Where one or more ridges 615 are present along the sides of the angled surfaces 616, the tactile feedback of the micropigmentation tool may be improved and the tool and a user's hand may be steadier at those given angles and desired angles.

It should be understood that the injector guard 600 may include one or more ridges 615, may include one or more angled surfaces 616, may include both ridges 615 and angled surfaces 616, or may include neither, without deviating from the scope or intent of this disclosure.

FIG. 20 shows an alternate embodiment of the injector guard 700. In this embodiment, the injector guard 700 includes one or more ribs 717 which extend at least partially along the height of the leg 710. The ribs 717 may extend from the foot 712 to the connection body 720, may extend only partly down from the connection body 720, or may extend only partly up from the foot 712. The ribs 717 may also or alternatively extend to the side of the leg 710 or at an angle, relative to the bottom of the foot 712, along the leg 710. The one or more ribs 717 may strengthen the leg 710 against flexion during use while also allowing for the leg 710 to be thin where thickness to the leg 710 might impede visibility of a user to the dermis or to make gripping of the micropigmentation tool easier or more comfortable. In the embodiment shown, the rib 717 thickens the leg 610 from the foot 712 to the connection body 720, allowing for improved resistance against flexion, while staying within the profile of the connection body 720 from a top-down perspective, thus allowing for a thinner leg 710 where the leg 710 extends beyond the connection body 720 from a top-down perspective.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention. Accordingly, various modifications, adaptations, combinations, and alternatives may occur to one skilled in the art without departing from the spirit of the invention and scope of the claimed coverage.

1. An injector guard, comprising:
a leg having a first top end and a first bottom end and defining a first axis from the first top end to the first bottom end; and
a connection body having a first top surface, a first bottom surface, and a first outer surface;
wherein the connection body is configured to connect to a component of a pigmentation device, and further wherein the pigmentation device further comprises a needle, the needle defines a second axis, the needle comprises a penetrating tip with a second bottom end which is distal from the first bottom surface, the connection body defines a third axis extending between the first top surface and the first bottom surface, and the leg at least partially extends away from the first bottom surface at least partially along the first axis, and further wherein the leg is connectable to the pigmentation device by means of the connection body, and further at least a portion of the leg is configured to be pressed against a dermis of an individual while enabling application of micropigmentation at a desired depth.

2. The injector guard according to claim 1, further wherein the component is disposable.

3. The injector guard according to claim 1, further wherein the component is reusable.

4. The injector guard according to claim 1, further wherein the component is an ink cartridge.

5. The injector guard according to claim 1, further wherein the component is a reusable tattoo gun.

6. The injector guard according to claim 1, further wherein the first bottom end comprises a second bottom surface which is closer to the first bottom surface than the second bottom end.

7. The injector guard according to claim 6, further wherein the leg extends at least partially away from the first outer surface relative to the third axis.

8. The injector guard according to claim 6, further wherein the leg extends at least partially towards the third axis relative to the first outer surface.

9. The injector guard according to claim 6, further wherein the connection body is substantially circular in cross-section when viewed from the first top surface through the third axis.

10. The injector guard according to claim 6, further wherein the first outer surface defines a first opening configured to enable the connection body to deform to allow at least part of the component to pass through the outer surface.

11. The injector guard according to claim 6, further wherein the connection body further comprises an inner surface, and further wherein the inner surface comprises a protrusion configured to resist motion of the injector guard relative to the component along the second axis.

12. The injector guard according to claim 6, further wherein the connection body further comprises an inner surface, and further wherein the inner surface comprises an at least partial thread feature configured to resist motion of the injector guard relative to the component along the second axis.

13. The injector guard according to claim 6, further wherein the leg is wider when viewed through the first axis proximate to the first bottom end than proximate to the first top end.

14. The injector guard according to claim 6, further wherein the leg comprises at least one rib feature which is wider when viewed through the first axis than portions of the leg which do not include the at least one rib, and further wherein the at last one rib is configured to at least partially resist flexion of the leg relative to the first axis.

15. A micropigmentation device, comprising:
an injector guard;
an ink cartridge; and
a tattoo gun;
wherein the injector guard further comprises a leg having a first top end and a first bottom end and defining a first axis from the first top end to the first bottom end, the ink cartridge further comprises a needle, the needle comprises a second top end, the needle comprises a penetrating tip with a second bottom end, the needle defines a second axis extending between the second top end and the second bottom end, the first axis and the second axis are substantially parallel, and the leg second bottom end is further away from the first top end than the first bottom end relative to the second axis, and further wherein the leg is connected to the pigmentation device by means of a connection body, and further at least a portion of the leg is configured to be pressed against a dermis of an individual while enabling application of micropigmentation at a desired depth.

16. The micropigmentation device according to claim 15, further wherein the injector guard is unitary with the ink cartridge.

17. The micropigmentation device according to claim 15, further wherein the injector guard is unitary with the tattoo gun.

18. A method of using a pigmentation device, comprising:
attaching an ink cartridge to a tattoo gun;
pressing an injector guard up to the dermis of an individual; and
tattooing the individual with the pigmentation device;
wherein the injector guard comprises a leg having a first top end and a first bottom end and defining a first axis from the first top end to the first bottom end, the ink cartridge further comprises a needle, the needle comprises a second top end, the needle comprises a penetrating tip with a second bottom end, the needle defines a second axis extending between the second top end and the second bottom end, the first axis and the second axis are substantially parallel, and the leg second bottom end is further away from the first top end than the first bottom end relative to the second axis, and further wherein the leg is connectable to the pigmentation device by means of a connection body.

19. The method of using a pigmentation device according to claim 18, further comprising attaching the injector guard to the ink cartridge.

20. The method of using a pigmentation device according to claim 18, further comprising attaching the injector guard to the tattoo gun.
